(19) United States
(12) Patent Application Publication
(10) Pub. No.: US 2009/0105547 A1
(43) Pub. Date: Apr. 23, 2009

(54) Adjustable Retractor Blade

(75) Inventors: Alex Vayser, Mission Viejo, CA (US); David M. Giuntoli, San Marcos, CA (US); Thomas L. Grey, San Marcos, CA (US)

Correspondence Address: Crockett & Crockett, Suite 400, 24012 Calle de la Plata, Laguna Hills, CA, 92653, US

(73) Assignee: Spotlight Surgical, Inc.

(21) Appl. No.: 12/107,006

(22) Filed: Apr. 21, 2008

Related U.S. Application Data
(60) Provisional application No. 60/925,461, filed on Apr. 20, 2007.

(51) Int. Cl. A61B 1/32

(52) U.S. Cl. 600/228

(57) ABSTRACT

An adjustable retractor blade may include a vertical blade portion integrated with an adjustable attachment portion, which is used to attach the blade to a support arm or frame. The adjustable portion is compatible with existing retractor support arms or frames and allows the vertical blade's position within the body to be easily adjusted without the need for an assistant.

### RELATED APPLICATIONS

This application claims priority from copending U.S. provisional patent application 60/925,461 filed Apr. 20, 2007.

### FIELD OF THE INVENTIONS

The inventions described below relate to the field of medical tools and more specifically, to adjustable retractor blades for use in surgery, particularly spine surgery.

### BACKGROUND OF THE INVENTIONS

Surgical procedures often require the use of retractors to pull apart and hold tissue to expose the underlying tissue on which the surgery is to be performed. Blade retractors are a type of retractor that typically have a flat or slightly curved vertical blade portion that is put into the body. The blade may have a handle portion that is used to manipulate the blade or may be attached to a frame. One or more blade retractors may be used in a surgical procedure. The frame typically allows the blades to be adjusted away from each other to open the surgical wound.

Surgeons often need to adjust the position of the blade portion to improve visualization of and access to an anatomical structure or to view or access nearby structures. In particular, it is often necessary to adjust the angular position in the plane corresponding to the side view of the vertical portion. This is sometimes referred to as “toe in”, causing the tip of the blade to go further into the tissue. Also, it is often necessary to adjust the blade's angular position in the plane corresponding to the front view of the vertical portion. This is sometimes referred to simply as “angle”, causing the end of the blade to move, for example, relative to the patient's spine from one spinous process to an adjacent spinous process. Less often, the angular position in the plane corresponding to the top view may be adjusted. This is sometimes referred to as “swivel”, causing the width of the blade to rotate about its vertical axis. Often, some combination of toe-in and angle adjustment is preferred. Hand held blades are easily manipulated, but require an assistant to manipulate the blade under the direction of the surgeon. Frame mounted blades have very limited adjustment capability. Prior art devices have attempted to provide some position adjustment capability, but invariably the adjustment is made via complicated clamps along the frame and not directly on the blade itself. Such prior art devices require an assistant to loosen the clamps, then the surgeon repositions the blades, and then the assistant tightens the clamps.

Existing retractor blades typically include a vertical blade portion and an attachment portion, which is used to attach to a frame. Nonadjustable or limited adjustable retractor blade systems exist, such as McCulloch or Caspar blade systems shown in FIG. 1. McCulloch systems have a square hole in the attachment portion that fits on a square arm on the frame. The square shape prevents any adjustment. Caspar systems have a head on the attachment portion, said head may be attached to a handle to adjust the angle, but not toe-in, and the angle adjustment is possible only because of hinges placed in the arm of the frame. Blade retractors are typically made from a suitable metal, such as steel, aluminum or titanium, so that they provide the strength needed to retract tissue and so that they may be cleaned and resterilized, typically by steam sterilization or autoclave process, for repeated use.

What is needed is a frame-mounted blade that provides adjustment of the blade's position in space after it has been positioned in the body, said adjustment being able to be accomplished directly by the surgeon without the aid of an assistant.

### SUMMARY

An adjustable retractor blade may include a vertical blade portion integrated with an adjustable attachment portion, which is used to attach the blade to a support arm or frame. The adjustable portion is compatible with existing retractor support arms or frames and allows the vertical blade's position within the body to be easily adjusted without the need for an assistant.

An adjustable retractor blade has a vertical blade portion, but has position adjustment capability built into the attachment portion. Such adjustment is made by using one or two tools to position the blade to a desired orientation and then fix that position using suitable fixing means. In other embodiments, the adjustment means is self-retaining such that the surgeon simply adjusts the position without requiring a separate tightening step. The adjustable retractor blade is fully compatible with existing frame designs and can be used concurrently with existing nonadjustable blades to meet the surgeon's need for tissue retraction during surgery.

An adjustable retractor blade includes a coupling mechanism providing one or more rotational axes of adjustment in addition to conventional lateral adjustment. In a first configuration an adjustable retractor blade may be rotated about the axis of the arm to which the retractor is clamped. The rotation may be controlled by frictional clamping, or controllable using gears, splines, cogs or other suitable mechanisms.

In another configuration, an adjustable blade retractor may be adjusted to rotate about two orthogonal axes of rotation in addition to lateral adjustment. The support arm to which the adjustable blade retractor is clamped may be one of the two axes of rotation.

In a yet another configuration, an adjustable blade retractor may be adjusted to rotate about three orthogonal axes of rotation in addition to lateral adjustment. The support arm to which the adjustable blade retractor is clamped may be one of the three axes of rotation.

An adjustable blade retractor may be used in a method of retracting tissue in a surgical field including the steps of creating an incision to expose the tissue of interest, and locating a retractor support arm with a plurality of splines arranged on the outer surface of the support arm adjacent the incision, and then inserting a retractor blade into the incision, the retractor blade having an attachment portion for engaging the retractor support arm splines at a pre-selected horizontal position along an axis, the attachment portion also engaging the retractor support arm at a pre-selected radial position about the axis, each pre-selected radial position from 1° to 5° apart, and then rotating the retractor blade to a selected radial position to create a surgical field, and then engaging the retractor blade to the retractor frame in the selected radial position at a selected horizontal position.

### BRIEF DESCRIPTION OF THE DRAWINGS

FIG. 1 is a perspective view of a prior art retractor blade and frame arm.

FIG. 2 is a perspective view of a blade retractor and frame arm that allows toe-in adjustment using a star shaped hole to engage a standard frame arm.

FIG. 2A is a close view of the side of the blade and frame arm of FIG. 2.

FIG. 3 is a perspective view of a blade retractor and frame arm that allows toe-in adjustment using matching splines.

FIG. 3A shows a close cross-section view of a portion of the blade and splined frame of FIG. 3 taken along A-A.

FIG. 4 is a perspective view of a blade retractor that allows toe-in adjustment using a shaft coupling.

FIG. 4A is a cross-section view of the blade retractor coupling of FIG. 4 taken along B-B.

FIG. 5 is a perspective view of an alternative shaft coupling retractor blade.

FIG. 6 is a perspective view of a blade retractor with toe-in adjustment using a worm gear.

FIG. 6A is a cross-section view of the blade retractor adjustment mechanism of FIG. 6 taken along C-C.

FIG. 7 is a perspective view of an adjustable blade retractor with adjustment for toe-in, angle and swivel using a combination of a ball and socket shaft coupling.

FIG. 7A is a cross-section view of the blade retractor of FIG. 7 taken along D-D.

FIG. 7B is a cross-section view of the blade retractor of FIG. 7 taken along E-E.

FIG. 8 is a cross-section view of an alternate adjustable retractor blade with toe-in, angle and swivel adjustment capability.

### DETAILED DESCRIPTION OF THE INVENTIONS

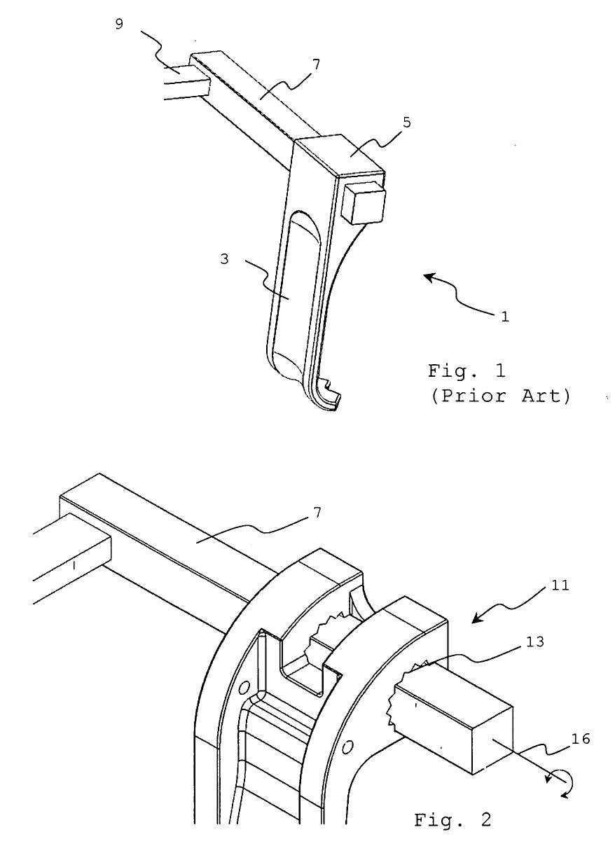

FIG. 1 shows a prior art retractor blade system 1, which is commonly known as a McCulloch retractor. Vertical blade portion 3 retracts tissue. Attachment portion 5 has a square shaped cutout that engages square arm 7, which is typically integral with a frame 9. The square shaped engagement prevents the retractor blade from rotating about the arm.

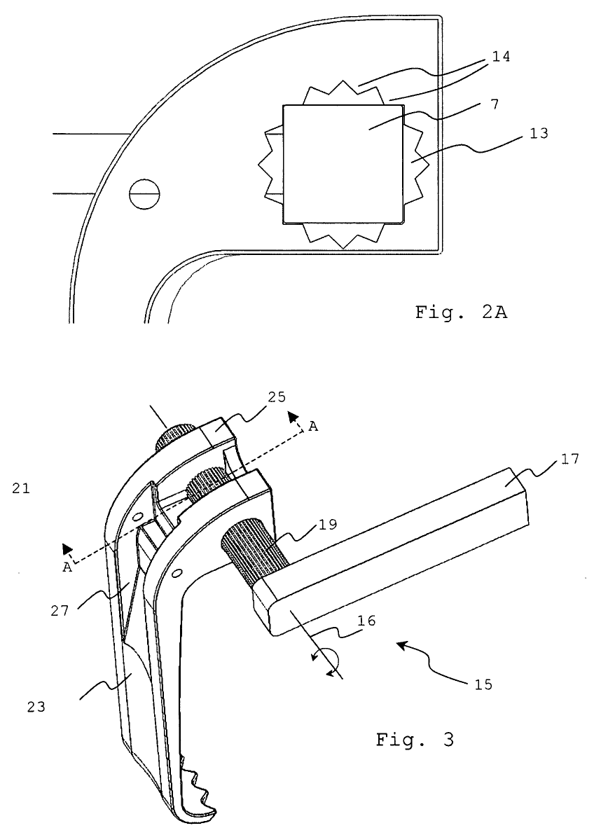

FIG. 2 illustrates an adjustable blade retractor system 11 that works with the prior art square shaped arm 7. Star shaped engagement channel 13 in the retractor allows for toe-in or radial adjustment of the retractor about first rotational axis 16. The retractor is removed from the arm, the retractor as radially adjusted, toe-in, about the rotation axis, and the retractor is replaced, engaging a different set of teeth 14 in star shaped engagement channel 13.

FIG. 2a shows a close-up side view of this embodiment showing how arm 7 engages teeth 14 in star shaped engagement channel 13. In this configuration, radial or angular rotation, toe-in, is in steps of 22.5 degrees. Any suitable radial or angular rotation increment less than 45° may be used. Smaller rotation increments reduces the amount of force that each tooth can withstand resulting in the potential for the arm to strip the teeth and for the retractor to slip.

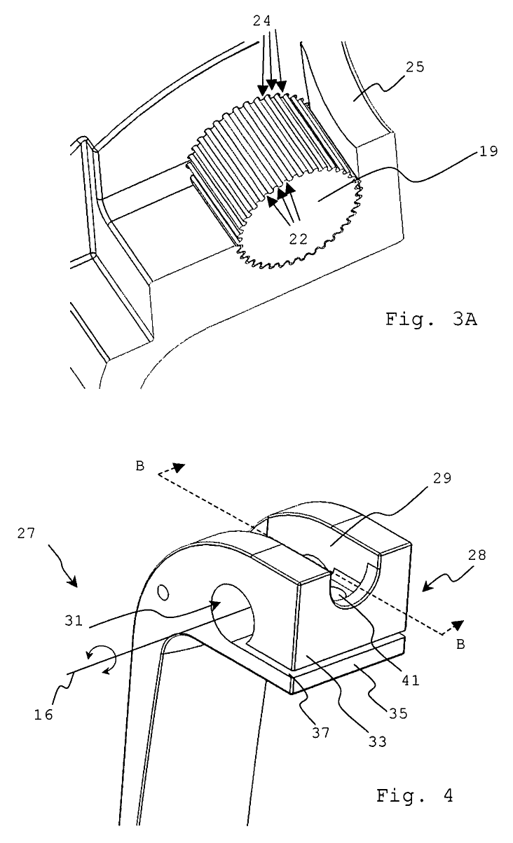

FIG. 3 illustrates an adjustable blade retractor system 15 including frame 17, arm 19 and retractor blade 21. Retractor blade 21 further includes vertical blade portion 23 and attachment portion 25. Retractor blades such as retractor blade 21 may include one or more accessory engagement areas, such as cutout 27, for attaching accessories, such as an illumination device or any other suitable accessory. Frame 17 may include one or more retractor engagement arms such as arm 19 and the engagement arms may be moveable relative to each other using a rack and pinion mechanism or similar movement mechanism. Frame 17 may also be formed in any suitable shape, such as a circle or square such that one or more engagement arms may be suitably spaced along its length.

Splines 22 of arm 19 and splines 24 of adjustment portion 25 permit adjustment of toe-in first rotational axis 16 by removing the retractor and attachment portion 25 from arm 19, repositioning the vertical blade portion 23, which causes splines 24 in attachment portion 25 to be rotated to a new position relative to spines 22 of arm 19, then reengaging attachment portion 25 onto arm 19 such that splines 22 on arm 19 engage the repositioned splines 24 in attachment portion 25. Typically, frame 17 and arm 19 are fixed in position relative to a surgical site, providing the force necessary to support retractor blade 21 in its new position. Often, retractor blades may be arranged in opposite pairs providing complementary support force to each other via the frame assembly.

The angular resolution available is a function of spline design. The design shown provides approximately 10 degrees of angular resolution set by the spread of the splines 22 around the circumference of arm 19, for example, 360 degrees divided by 36 splines produces 10 degrees of rotation per spline on the arm. The preferred angular resolution is 1 to 5 degrees, but the resolution is limited by the strength of the material used and the ability of smaller splines to have enough surface area to provide enough supporting force before the splines fail and the arm or engagement mechanism strips, much like the thread on a bolt can strip if the bolt is tightened too much. Metal is the preferred material, but strength reinforced plastics or ceramics may also be suitable.

FIG. 3A shows a close cross-section view of attachment portion 25. Splines 22 of arm 19 engage splines 24 of attachment portion 25.

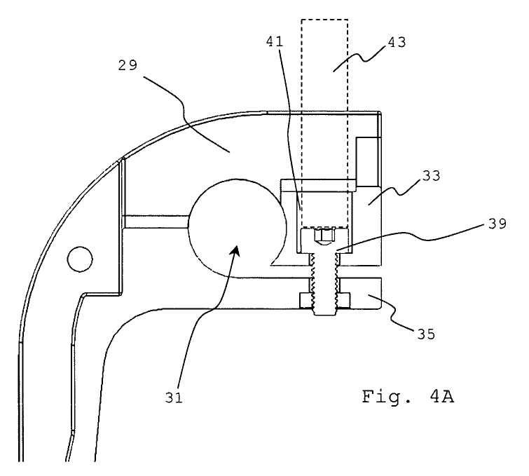

FIG. 4 shows an adjustable blade retractor 27 that includes shaft coupling clamp mechanism 28. The retractor may have a cutout 29 that is suitable for attachment of any suitable accessory, such as an illumination device. A suitable frame arm may be inserted into channel 31. In this example, the arm is round and of a diameter slightly smaller than channel 31 although any suitable geometry may be used. Upper clamping portion 33 and lower clamping portion 35 are in spaced relation to each other such that a gap 37 exists between them, gap 37 extends to channel 31. The two clamping portions may be brought toward each other using a suitable means, such as a screw or cam device, causing channel 31 to engage the support arm. Friction then prevents the retractor from rotating about the arm, thereby allowing retractor's angular position to be fixed.

FIG. 4A is a cross-section view through B-B of adjustable blade retractor 27 of FIG. 4 illustrating detail of shaft coupling clamp 28. Screw 39 is used to tighten upper clamping portion 33 toward lower clamping portion 35 to generate the clamping force on an arm inside channel 31. Screw 39 may engage threads cut directly into lower clamping portion 35 or may engage a nut preferably recessed into lower clamping portion 35. Screw 39 has been recessed into hole 41 which provides sufficient side wall for tool 43 to be inserted and used as a pry to rotate the retractor blade about first rotational axis 16 to adjust toe-in. Tool 43 may be affixed with an end that matches the screw, for example a hex end to match a hex head screw, so that the tool could be used to tighten the clamping portions and hold the retractor blade fixed in the desired angular position.

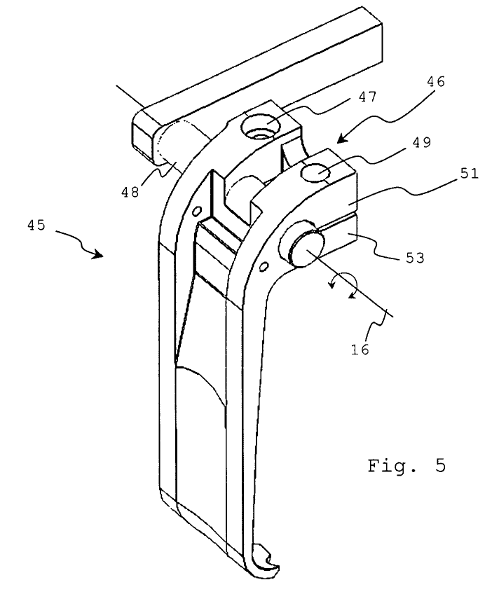

FIG. 5 illustrates an adjustable retractor blade 45 having an alternative shaft coupling clamp mechanism 46. In this configuration, screw hole 47 is provided separately from pry tool hole 49. Pry tool hole 49 preferably extends through upper clamp portion 51 and at least partially into lower clamp portion 53 to maximize the prying force. Pry tool hole 49 is preferably made to go all the way through lower clamping portion 53 to facilitate cleaning for subsequent resterilization after use. In this case, pry tool hole 49 preferably alters shape as it goes through lower clamping portion 53, for example as a cone, to provide a mechanical stop for the pry tool so that the pry tool cannot go all the way through the hole and potentially injure the patient. Adjustable retractor blade 45 may be made to rotate about first rotational axis 16 centered in support arm 48.

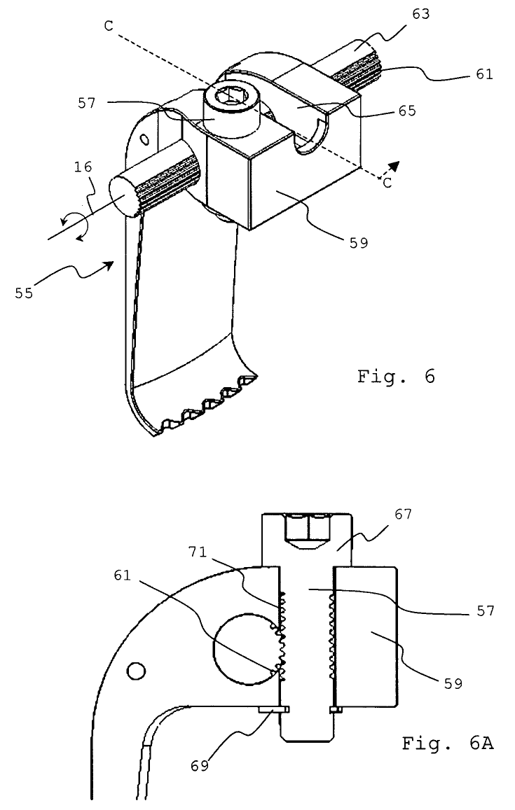

FIG. 6 illustrates adjustable retractor blade 55 with worm gear 57 located inside of attachment portion 59. Threads 71 of worm gear 57 engage the splines 61 of arm 63, which is typically attached to a frame as previously discussed. A rotation tool may temporarily engage head 67 of worm gear 57, such as a hex head tool. Other suitable rotation mechanisms can be fixed or made integral, temporarily or permanently, with the worm gear. As worm gear 57 is rotated, threads 71 engage splines 61 and cause retractor blade 55 to rotate about first rotational axis 16 in arm 63. The advantage of a worm gear is that it is designed to be self-retaining, such that when the rotation is stopped, the retractor blade maintains its new position and does not rotate back into its starting position. Very small angular adjustments are possible. Worm gear 57 is shown placed to the side of accessory cutout 65, but may be placed in any other suitable position in attachment portion 59 if no such cutout is required.

Referring now to FIG. 6A, worm gear 57 is secured in attachment portion 59 by head 67 and retaining ring 69 such that worm gear 57 is allowed to rotate freely. Threads 71 engage splines 61.

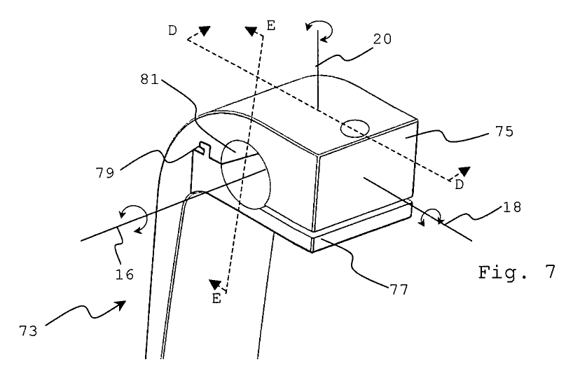

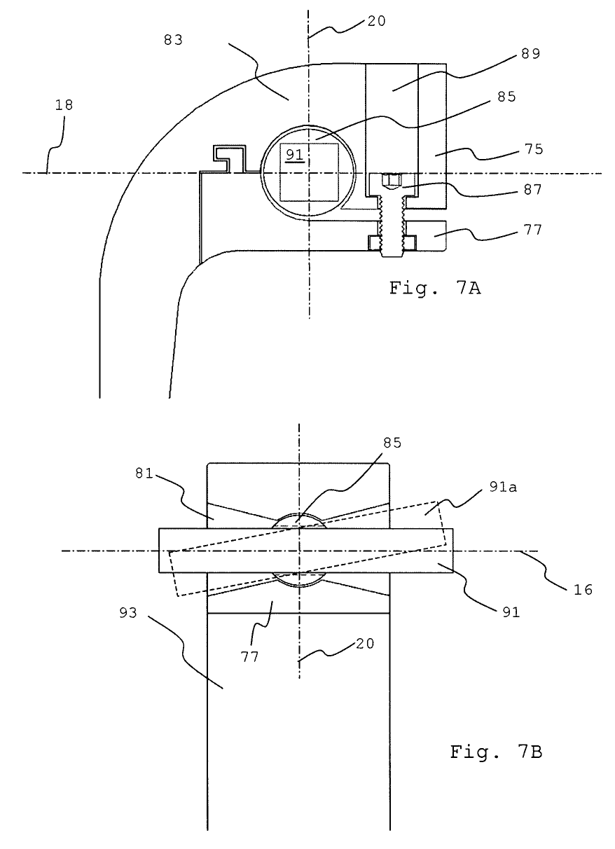

Adjustable retractor blade 73 of FIGS. 7, 7A and 7B may be rotated about first rotational axis 16, second rotational axis 18 and third rotational axis 20 to provide toe-in, angle, and swivel adjustment capabilities. It employs a clamping mechanism similar to that in FIG. 4. Upper clamping portion 75 is integral with the body of the retractor blade, but lower clamping portion 77 is releasably attached to the body of the retractor as shown by tongue and groove joint 79. In this configuration, tongue and groove joint 79 is designed to allow lower clamping portion 77 to partially hinge, and it may be replaced by a full hinge joint with a pin. Alternatively, lower clamping portion 77 may be releasably secured to upper clamping portion 75 using other suitable techniques, such as one or more screws. Channel 81 is actually conical in shape with the wider portion on the outside as shown.

At or near the center of attachment portion 83 of the retractor blade, a spherical cutout is made that fits spherical adapter 85. In this configuration, spherical adapter 85 is shown with a square hole cut all the way through it to allow a square shaped arm such as arm 91 to be engaged into the hole, but virtually any arm geometry can be accommodated. Because of the square shape engagement, spherical adapter 85 cannot rotate about the arm, it can only slide along the arm. Means may be provided to limit or prevent this sliding motion, for example, spring-loaded ball and detent or a setscrew, which may be set by providing an access hole through the retractor blade at a suitable location. Spherical adapter 85 is trapped by upper clamping portion 75 and releasable lower clamping portion 77. In this configuration, screw 87 is used to force the clamping portions together, thereby applying a clamping force on spherical adapter 85 that prevents rotation. Hole 89 is provided for a tool similar to that described in FIG. 4A, although other suitable clamping means would be acceptable.

For example, the clamping and prying means may be separated as discussed for FIG. 5. In the configuration of FIG. 7A, the user inserts the tool into hole 89, loosens screw 87, uses the tool to adjust the angular position of the retractor blade about one or more of the rotational axes and then uses the tool to tighten screw 87 thereby fixing the retractor blade's position, and removes the tool. Adjustment can be made at any time in a surgical procedure.

FIG. 7B is a cross-sectional view along line E-E in FIG. 7, showing how angular rotation about rotational axis 18 is achieved. Arm 91 is shown engaged inside spherical adapter 85. The conical shape of hole 81 allows arm 91 to rotate within hole 81 as shown by arm position 91a. In practical use, arm 91 is fixed to a frame that does not move, so the end result is that retractor blade 93 is allowed to rotate about rotational axis 18 to different angular positions relative to arm 91. Angular adjustment of toe-in, angle and swivel or any combination thereof is possible. Since hole 81 is conical, angular rotation is possible in any direction, limited in extent only by the size of hole 81, which can be made larger to allow a larger maximum angle of adjustment or made smaller to limit the maximum angle of adjustment. Other configurations using the concept of the center spherical adapter may also be used.

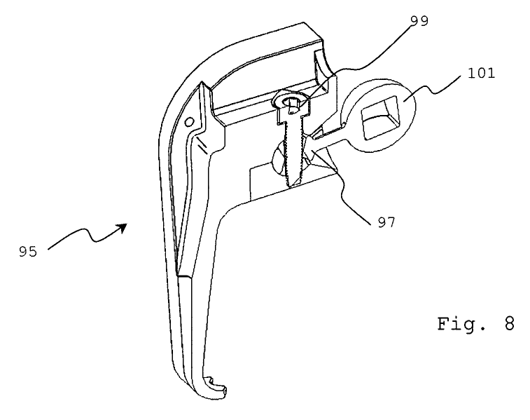

For example, FIG. 8 illustrates a cross-section view of adjustable retractor blade 95 in which spherical adapter portion 97 has been designed to allow rotation about clamping screw 99. In this configuration, spherical adapter portion 97 includes arm adapter portion 101, which can be made to adapt to any support arm or frame and may include separate locking (e.g., set screw) or clamping features to attach to the support arm or frame.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A surgical retractor comprising:
a retractor support arm with a plurality of splines arranged on the outer surface of the support arm;
a retractor blade having an attachment portion for engaging the retractor support arm splines at a pre-selected horizontal position along an axis, the attachment portion also engaging the retractor support arm at a pre-selected radial position about the axis, each pre-selected radial position from 10 to 5° apart.

2. The surgical retractor of claim 1 further comprising:
means for clamping the retractor blade to the retractor support arm.

3. The surgical retractor of claim 2 wherein the means for clamping is frictional.

4. The surgical retractor of claim 1 further comprising:
a retractor support arm with a plurality of cogs arranged on the outer surface of the support arm;
a retractor blade having an attachment portion for engaging the retractor support arm cogs at a pre-selected horizontal position along an axis, the attachment portion also engaging the retractor support arm at a pre-selected radial position about the axis, each pre-selected radial position from 1° to 5° apart.

5. The surgical retractor of claim 1 further comprising:
a retractor support arm with a plurality of gear teeth arranged on the outer surface of the support arm;
a retractor blade having an attachment portion for engaging the retractor support arm gear teeth at a pre-selected horizontal position along an axis, the attachment portion also engaging the retractor support arm at a pre-selected radial position about the axis, each pre-selected radial position from 1° to 5° apart.

6. A method of retracting tissue in a surgical field comprising the steps:
creating an incision to expose the tissue of interest;
locating a retractor support arm with a plurality of splines arranged on the outer surface of the support arm adjacent the incision;
inserting a retractor blade into the incision, the retractor blade having an attachment portion for engaging the retractor support arm splines at a pre-selected horizontal position along an axis, the attachment portion also engaging the retractor support arm at a pre-selected radial position about the axis, each pre-selected radial position from 1° to 5° apart;
rotating the retractor blade to a selected radial position to create a surgical field; and
engaging the retractor blade to the retractor frame in the selected radial position at a selected horizontal position.

7. A surgical retractor comprising:
a retractor support arm adjacent a surgical field;
a retractor blade having an attachment portion for engaging the retractor support arm at a pre-selected horizontal position along an axis, the attachment portion also engaging the retractor support arm at a pre-selected toe-in position about the axis, each pre-selected toe-in position 45° or less apart.

8. The surgical retractor of claim 1 wherein each pre-selected toe-in position 22.5° or less apart
